(19) United States
(12) Patent Application Publication
(10) Pub. No.: US 2026/0086076 A1
(43) Pub. Date: Mar. 26, 2026

(54) FECAL GAS TIME CURVE ANALYSIS METHOD FOR BRISTOL STOOL TYPE CLASSIFICATION

(71) Applicant: HEM PHARMA INC., Pohang-si (KR)

(72) Inventors: Yo Sep JI, Suwon-si, Gyeonggi-do (KR); Eun Sung JUNG, Suwon-si (KR); Young Chun PARK, Pohang-si (KR); Ju Young PARK, Pohang-si (KR)

(21) Appl. No.: 19/408,578

(22) Filed: Dec. 4, 2025

Related U.S. Application Data
(63) Continuation of application No. PCT/KR2024/007776, filed on Jun. 7, 2024.

(30) Foreign Application Priority Data
Jun. 7, 2023 (KR) 10-2023-0073106

(51) Int. Cl. G01N 33/00; G01N 33/483

(52) U.S. Cl. CPC G01N 33/0054; G01N 33/0042; G01N 33/0044; G01N 33/0047; G01N 33/4833

(57) ABSTRACT

Provided is a fecal gas analysis method for Bristol stool type classification, and more particularly, to a fecal gas time curve analysis method capable of classifying Bristol stool types based on a rate of gas concentration decrease for each stool sample. The fecal gas time curve analysis method for Bristol stool type classification enables classification of Bristol stool types regardless of gender, age, diet, or stool weight, and also allows identification of an individual's dietary habits. Therefore, the analysis method can be applied to personal health monitoring based on Bristol stool type classification.

### TECHNICAL FIELD

The present disclosure relates to a fecal gas analysis method for Bristol stool type classification and body condition analysis, and more particularly, to a fecal gas time curve analysis method capable of classifying Bristol stool types and analyzing body conditions based on a rate of gas concentration decrease over time for each stool sample.

The present application claims the benefit of priority to Korean Patent Application No. 10-2023-0073106 filed on Jun. 7, 2023 and incorporated herein by reference in its entirety.

### BACKGROUND

Due to changes in dietary habits and various stress factors in modern life, gastrointestinal diseases are on the rise. In order to determine the progression and causes of such gastrointestinal diseases, it is important to check the condition of stool, which is the result of daily food digestion.

Although it would be ideal for an individual to directly observe the condition of their stool after defecation, many people are reluctant to do so, and it is difficult for an individual to consistently monitor information, such as defecation time, frequency, and odor.

Another method for checking the condition of stool is to use a fecal gas. In this case, the absolute concentration of gases emitted directly from the stool may be measured by one or more gas sensors to assess the condition of the stool. However, the absolute gas concentration values vary depending on measurement conditions and environmental factors, which results in reduced accuracy.

As examples of techniques developed for fecal gas analysis, an analysis system, a fecal odor gas analysis system, and an exhaled gas analysis system have been disclosed (Japanese Patent Laid-Open Publication No. 2020-187074). However, in relation to fecal gas analysis, a need remains for more accurate and effective analytical methods through further development and diverse research.

Accordingly, the present inventors have made extensive efforts to develop a fecal gas analysis method for Bristol stool type classification. As a result, they have completed the present disclosure by discovering a fecal gas time curve analysis method capable of classifying Bristol stool types based on a rate of gas concentration decrease over time for each stool sample.

### DISCLOSURE OF THE INVENTION

### Problems to be Solved by the Invention

The present disclosure relates to a fecal gas analysis method for Bristol stool type classification and body condition analysis, and is conceived to provide a fecal gas time curve analysis method capable of classifying Bristol stool types and analyzing body conditions based on a rate of gas concentration decrease over time for each stool sample.

However, the problems to be solved by the present disclosure are not limited to the above-described problems. Although not described herein, other problems to be solved by the present disclosure can be clearly understood by a person with ordinary skill in the art from the following descriptions.

### Means for Solving the Problems

A first aspect of the present disclosure provides a method for analyzing fecal gas time curves of the same type for Bristol stool type classification and body condition analysis.

A second aspect of the present disclosure provides a fecal gas time curve analysis method for Bristol stool type classification and body condition analysis.

A third aspect of the present disclosure provides a fecal gas time curve output method for Bristol stool type classification and body condition analysis.

### Effects of the Invention

According to an embodiment of the present disclosure, the fecal gas time curve analysis method for Bristol stool type classification and body condition analysis enables classification of Bristol stool types regardless of gender, age, diet, or stool weight, and also allows identification of an individual's dietary habits. Therefore, the analysis method can be applied to personal health monitoring based on Bristol stool type classification.

### BRIEF DESCRIPTION OF THE DRAWINGS

FIG. 1 is a diagram illustrating the Bristol stool scale.

FIG. 2A to FIG. 2C are diagrams illustrating gas concentration graphs based on outputs from seven sensors and a total gas concentration graph (FIG. 2C) obtained by summing the real-time measurement values from the seven sensors when gas from a single stool sample was measured.

FIG. 3 is a diagram showing a gas concentration graph for each of the seven sensors or a total gas concentration graph for the seven sensors.

FIG. 4A to FIG. 4E are diagrams illustrating a process of converting the total gas concentration graphs of five Type 4 stool samples into time curves, arranging them in consideration of measurement time points after stool collection (Table 1), and checking whether they overlap into a single time curve for Type 4 stool.

FIG. 5 shows the expected inherent and complete time curve for Type 4 stool where the concentration of fecal gas measured immediately after stool collection in a toilet is set to 100%.

FIG. 6A and FIG. 6B are diagrams showing individual gas concentration graphs based on outputs from VOCs and ETO sensors among the seven sensors of FIG. 2A to FIG. 2C when five Type 4 stool samples were measured, and FIG. 6C is a diagram showing a single overlapped time curve for Type 4 stool based on outputs from the sensors through the process of FIG. 4A to FIG. 4E.

FIG. 7A and FIG. 7B are diagrams showing time curves for five Type 4 stool samples, and FIG. 7C is a graph obtained through the process of FIG. 4A to FIG. 4E to verify whether the five time curves overlap into a single time curve.

FIG. 8 is a graph obtained through the process of FIG. 4A to FIG. 4E to verify whether three time curves for Type 2 stool samples overlap into a single time curve.

FIG. 9A and FIG. 9B are diagrams showing time curves for five Type 6 stool samples, and FIG. 9C is a graph obtained through the process of FIG. 4A to FIG. 4E to verify whether the five time curves overlap into a single time curve.

FIG. 10 is a graph obtained through the process of FIG. 4A to FIG. 4E to verify whether three time curves for Type 6 stool samples not associated with consumption of spicy food overlap into a single time curve.

FIG. 11 is a graph obtained through the process of FIG. 4A to FIG. 4E to verify whether two time curves for Type 6 stool samples associated with consumption of spicy food overlap into a single time curve.

FIG. 12 is a diagram showing curve fitting performed to obtain a graph equation for the overlapped time curves for the five Type 4 stool samples shown in FIG. 7A and FIG. 7B.

FIG. 13A to FIG. 13G are diagrams illustrating a process of deriving the inherent and complete time curve for Type 4 stool of FIG. 5 through the process of FIG. 12.

FIG. 14 shows the inherent and complete time curves for stool types, including Type 2, Type 4, Type 6 without consumption of spicy food, and Type 6 with consumption of spicy food.

### BEST MODE FOR CARRYING OUT THE INVENTION

Hereafter, examples will be described in detail with reference to the accompanying drawings so that the present disclosure may be readily implemented by a person with ordinary skill in the art. However, it is to be noted that the present disclosure is not limited to the examples but can be embodied in various other ways. In the drawings, parts irrelevant to the description are omitted for the simplicity of explanation, and like reference numerals denote like parts throughout the whole document.

Throughout the whole document, the term “on” that is used to designate a position of one component with respect to another component includes both a case that the one component is adjacent to the other component and a case that any other component exists between these two components.

Further, throughout the whole document, the term “comprises or includes” and/or “comprising or including” used in the document means that one or more other components, steps, operation and/or existence or addition of elements are not excluded in addition to the described components, steps, operation and/or elements unless context dictates otherwise. Throughout the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Throughout the whole document, the term “step of” does not mean “step for.”

Throughout the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Throughout the whole document, a phrase in the form “A and/or B” means “A or B, or A and B.”

Hereafter, embodiments and examples of the present disclosure will be described in detail with reference to the accompanying drawings. However, the present disclosure may not be limited to the following embodiments, examples, and drawings.

A first aspect of the present disclosure provides a method for analyzing fecal gas time curves of the same type for Bristol stool type classification and body condition analysis.

According to an embodiment of the present disclosure, the method for analyzing fecal gas time curves of the same type for Bristol stool type classification and body condition analysis includes: a process (a) of measuring concentration values (ppm) of gases emitted from each stool sample using gas sensors; a process (b) of summing the measured gas concentration values in real time to derive a total gas concentration graph for each stool sample; a process (c) of setting a point at which the gas begins to decrease in the derived total gas concentration graph to 100% and deriving a time curve for each stool sample; a process (d) of arranging the derived time curves for the respective stool samples according to a sequence of measurement time points after stool collection; a process (e) of overlapping the time curves for the other stool samples based on a time curve for the stool sample measured first after stool collection to output a single time curve; and a process (f) of confirming that data of the stool samples of the same type overlap into a single time curve.

Throughout the whole document, the term “time curve” refers to a graph representing a rate (%) of gas decrease over time.

Throughout the whole document, the term “measurement time points after stool collection” refers to an elapsed time from stool collection in a toilet to the start of measurement in a laboratory.

Throughout the whole document, the term “time shifting” refers to arranging time curves to be analyzed according to a sequence of measurement time points after stool collection.

According to an embodiment of the present disclosure, the process (a) may include: a process of placing a prepared stool sample; a process of turning on a gas sensor switch to start data acquisition and turning on a fan within two minutes to circulate air inside the gas sensor; a process of placing the prepared stool sample on a stool collection paper within two minutes, closing a toilet lid to achieve maximum sealing from the outside, and attaching a connection tube to connect the toilet to the gas sensor; a process of measuring a fecal gas for 20 minutes to 40 minutes and then disconnecting the tube; and a process of turning off the gas sensor switch within two minutes to stop data transmission, but is not limited thereto.

During fecal gas measurement according to the present disclosure, the gas did not decrease for the first 5 to 10 minutes, and a constant signal was output. Therefore, the fecal gas was measured for more than 20 minutes to collect sufficient data to derive a time curve. When the fecal gas was measured for more than 40 minutes, a rate of decrease over time was too low to analyze the time curve. As the measurement time increased, the probability of being affected by the surrounding environment also rose. Accordingly, stool data were measured within 40 minutes.

A second aspect of the present disclosure provides a fecal gas time curve analysis method for Bristol stool type classification and body condition analysis. The features described above in respect of the first aspect of the present disclosure may equally apply to the method according to the second aspect of the present disclosure.

According to an embodiment of the present disclosure, the fecal gas time curve analysis method for Bristol stool type classification and body condition analysis includes: a process (a) of measuring concentration values (ppm) of gases emitted from each stool sample using gas sensors; a process (b) of summing the measured gas concentration values in real time to derive a total gas concentration graph for each stool sample; a process (c) of setting a point at which the gas begins to decrease in the derived total gas concentration graph to 100% and deriving a time curve for each stool sample; a process (d) of arranging the derived time curves for the respective stool samples according to a sequence of measurement time points after stool collection; a process (e) of overlapping the time curves for the other stool samples based on a time curve for the stool sample measured first after stool collection to output a single time curve; a process (f) of confirming that data of the stool samples of the same type overlap into a single time curve; a process (g) of performing curve fitting on the overlapped time curve for the stool samples of the same type, as confirmed above, to derive a time curve equation; a process (h) of deriving a single type of time curve, in which the amount of gas measured immediately after stool collection is set to 100%, through a calculation of

$$TIME CURVE EQUATION AS DERIVED BY ABOVE X \frac{100}{GAS AMOUNT ( \% ) AT 0 MINUTES IN THE TIME CURVE EQUATION} ;$$

and a process (i) of comparing the derived time curve with another type of time curve.

In the process (h), the “gas amount (%) at 0 minutes in the time curve equation” refers to a y-intercept of the time-curve equation derived in the process (g).

A third aspect of the present disclosure provides a fecal gas time curve output method for Bristol stool type classification and body condition analysis. The features described above in respect of the first and second aspects of the present disclosure may equally apply to the method according to the third aspect of the present disclosure.

According to an embodiment of the present disclosure, the fecal gas time curve output method for Bristol stool type classification and body condition analysis includes: a process (a) of measuring concentration values (ppm) of respective gases emitted from a single stool sample using gas sensors; a process (b) of deriving time curves for the respective gases by setting a point at which the measured gas concentration values begin to decrease to 100%; and a process (c) of arranging the derived time curves for the respective gases according to a sequence of measurement time points after stool collection.

Hereinafter, the present disclosure will be explained in more detail with reference to Examples. However, the following Examples are illustrative only for better understanding of the present disclosure, but do not limit the present disclosure.

### MODE FOR CARRYING OUT THE INVENTION

### Examples

### Example 1. Gas Sensor Setup and Sample Placement

### (1) Preparation of Gas Sensor

Gas sensors were connected to a power supply for stabilization. Then, a gas sensor array was configured to measure human breath in order to verify that the gas sensors were properly connected and functioning normally.

### (2) Preparation and Placement of Sample

After a stool collection paper was placed in a toilet, a switch was turned on to begin data acquisition from the gas sensors. After two minutes, a fan was turned on to allow outside air to be drawn into and circulated through the gas sensor array. After another two minutes, a prepared stool sample was placed on the stool collection paper, and a toilet lid was closed to create a sealed environment. A tube was attached to the toilet lid to connect the toilet to the gas sensor array, thereby allowing fecal gases from the toilet to be drawn into the sensor array. Fecal gases were measured for about 30 minutes. Then, the tube was disconnected. After two minutes, the switch of the gas sensors was turned off to stop data transmission.

### Example 2. Fecal Gas Measurement and Time Curve Output

### (1) Gas Measurement and Time Curve Output for Each Stool Sample

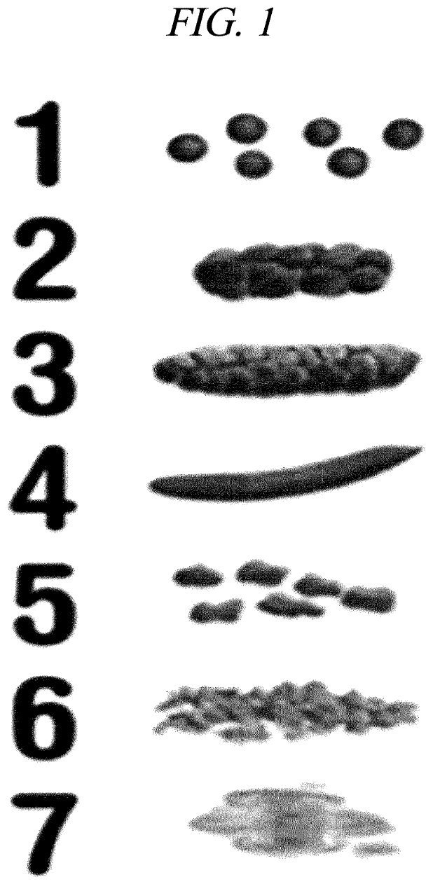

Fecal gas measurements were conducted on 13 stool samples under different conditions of Bristol stool type (see FIG. 1), diet, subject, age, gender, and stool weight. The stool samples were not measured immediately after stool collection in the toilet, and the fecal gas measurement test was carried out through the following processes.
Process 1. Defecate into a toilet and collect a stool sample in a stool container
Process 2. Transport the stool container to a laboratory
Process 3. Attach a stool collection paper to a toilet installed in the laboratory and place the collected stool sample on the stool collection paper
Process 4. Measure fecal gas data for 30 minutes

Since the location of the toilet (distance from the laboratory) and the time required to set up the test equipment varied in each test, an elapsed time from stool collection in the toilet to the start of measurement in the laboratory differed among the 13 stool samples. For example, a stool sample collected on the first day was measured 9 minutes after stool collection, whereas a stool sample collected on the second day was measured 30 minutes after stool collection, with each measurement lasting 30 minutes. The time from stool collection in the toilet to the start of measurement in the laboratory (“measurement time point after stool collection”) for all 13 stool samples is shown in Table 1 below.

TABLE 1

| | | Measurement time |
|---|---|---|
| Stool | | point after stool |
| type | Stool | collection [min] |
| 2Type | Subject B 2Type 55 g | 26 |
| | Subject B 1~2Type | 21 |
| | 43 g | |
| | Subject B 2Type 70 g | 13 |
| 4Type | Subject A 4Type 47 g | 29 |
| | Subject A 4Type | 24 |
| | 27.4 g | |
| | Subject A 4Type | 15 |
| | 22.7 g | |
| | Subject A 4Type | 10 |
| | 20.67 g | |
| | Subject E 4Type 46 g | 13 |
| 6Type | Subject A 6Type | 16 |
| | 23.01 g | |
| | Subject A | 9 |
| | 6Type110.06 g | |
| | Subject A 6Type | 12 |
| | 40.54 g | |
| | Subject C 6Type 67 g | 36 |
| | Subject C 6Type 86 g | 32 |

### Subject A: Female; Subject B: Female; Subject C: Male; Subject E: Male

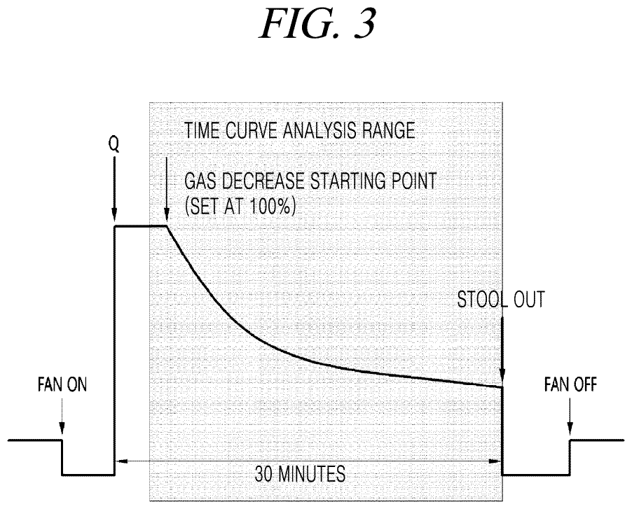

For measurement, each stool sample was placed on a stool collection paper attached to the toilet, a toilet lid was closed, and a tube was connected to measure fecal gases for about 30 minutes. Time curve analysis was conducted using SO₂, H₂S, NH₃, ETO, VOCs, CH₂O, and C₂H₄ gas sensors. In addition to the time curves obtained from individual sensors, a total gas concentration graph derived by summing the real-time outputs from all the seven sensors was used to analyze the time curves for the respective stool types (i.e., Both the gases measured individually by the seven gas sensors and the total sum of the gases were used). A point at which the gas began to decrease in each gas concentration graph or total gas concentration graph was set to 100%, and the time curve for each stool sample was derived accordingly (see FIG. 3).

Hereinafter, the term “time curve” refers to a graph showing a rate of gas decrease over time.

(2) Output of Single Type of Time Curve Using Total Gas Concentration Graph from Seven Sensors: Analysis of One Total Gas Concentration Graph Per Stool Sample

The seven sensors, including SO₂, VOCs, CH₂O, ETO, C₂H₄, H₂S, and NH₃ sensors, were used for time curve analysis. Both the individual gas concentration graphs from the respective sensors and the total gas concentration graph derived by summing the measurement values from all the seven sensors were used. Time curve analysis was conducted based on the total gas concentration graph derived by summing the measurement values from all the seven sensors in real time.

The time curve represents the rate (%) of gas decrease over time, and the concentration of fecal gas is highly time-sensitive. Thus, the time factor is crucial for the analysis. Therefore, for all the analyses, the time curves for the respective stool samples of the same type were arranged according to their “measurement time point after stool collection” (time shifting) and then overlapped for comparison.

The following describes the time curve analysis of Type 4 stool, and Type 2 and Type 6 stools were also analyzed in the same manner.

Five stool samples were classified as Type 4, and, thus, five time curves were obtained. These samples were collected on different days, and the subjects differed in diet, gender, and age. Details of the five Type 4 stool samples are shown in Table 2 below.

TABLE 2

| | | Order of stool | | | Measurement time |
|---|---|---|---|---|---|
| Stool | | collection and | Stool | | point after stool |
| type | Subject | measurement date | weight | | collection |
| 4 Type | Subject A | Day 1 | 47 | g | 29 min |
| | (Female in | Day 2 | 27.4 | g | 24 min |
| | 20s) | Day 3 | 22.7 | g | 15 min |
| | | Day 4 | 20.67 | g | 10 min |
| | Subject E | Day 5 | 46 | g | 13 min |
| | (Male in | | | | |
| | 20s) | | | | |

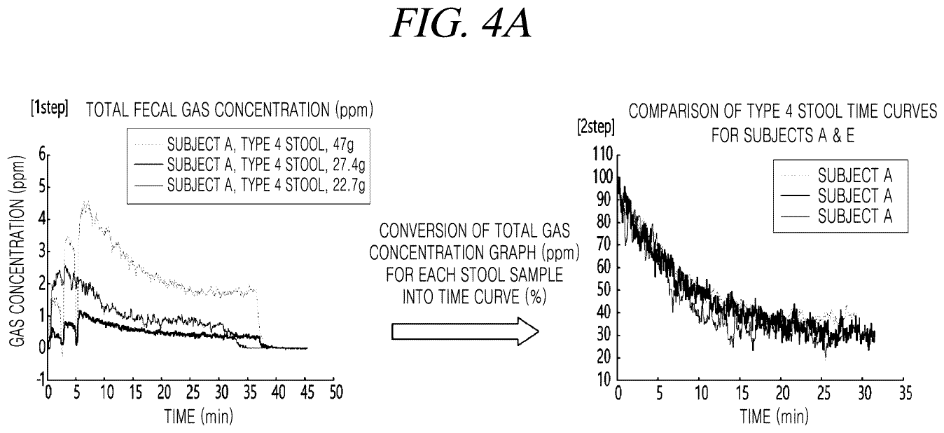

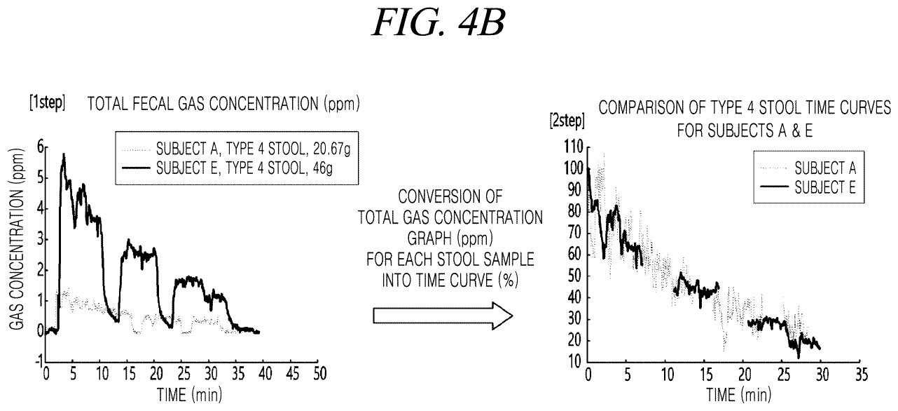

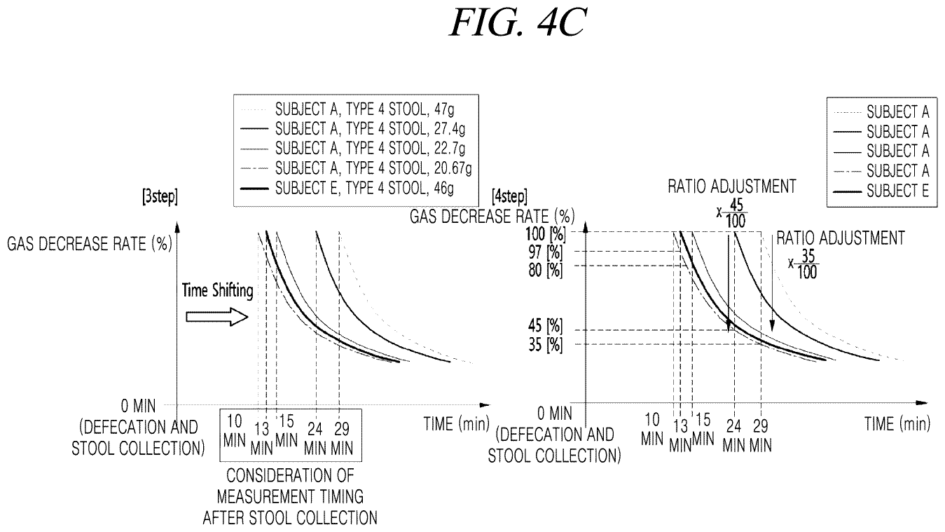

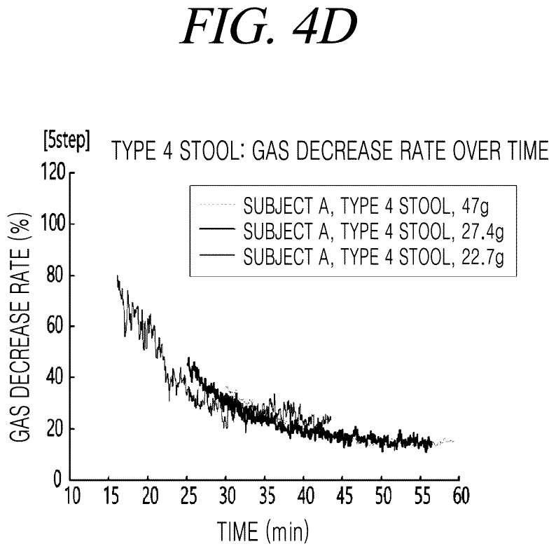

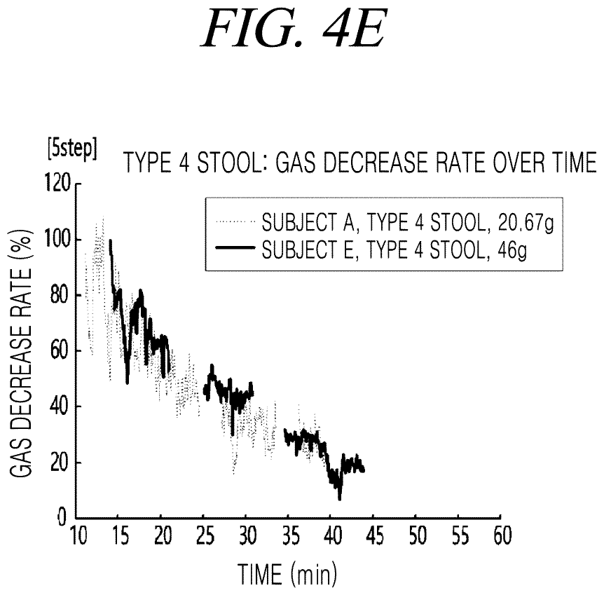

Hereafter, analytical processes for the five Type 4 stool time curves will be described (see FIG. 4A to FIG. 4E).
Process 1. Derive total gas concentration graphs for respective stool samples.

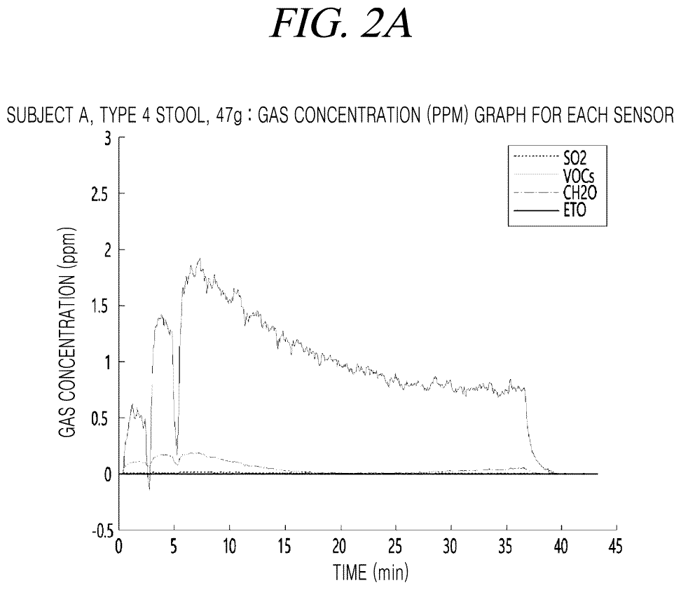

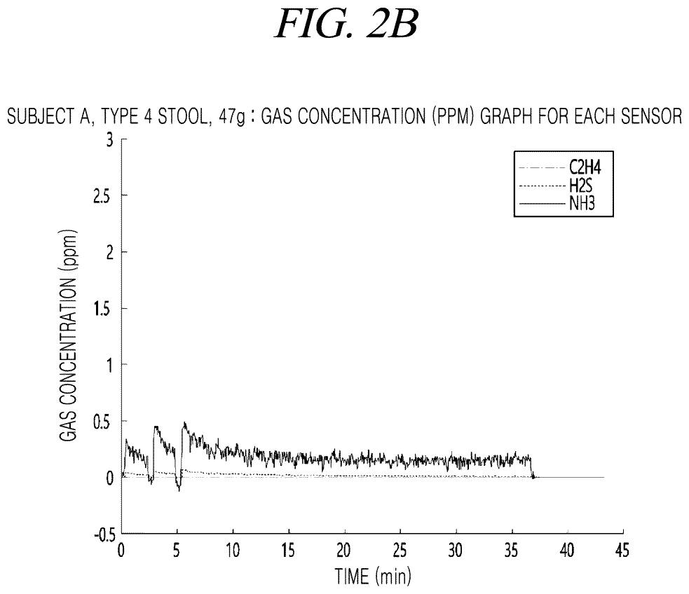

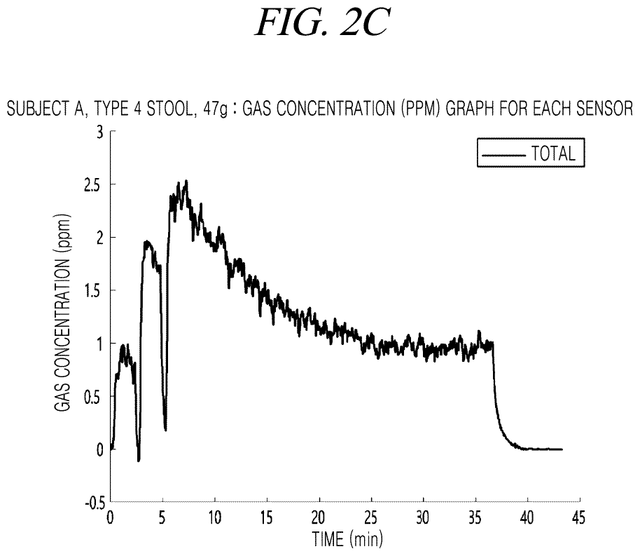

When a fecal gas from one stool sample was measured, gas concentration graphs were individually output from the seven sensors and then summed in real time to obtain one total gas concentration graph. That is, one total graph was analyzed for each stool sample (see FIG. 2C).
Process 2. Derive time curves for five stool samples by setting a point at which each gas begins to decrease in the total gas concentration graph to 100%: The five total gas concentration graphs from the five stool samples were converted into five time curves.
Process 3: Arrange the five time curves according to the measurement time point (0 min) after stool collection (time shifting) rather than the measurement date

The five time curves derived for the stool samples are time-shifted according to a sequence of measurement time points after the collection of each stool sample (see Table 2).
Process 4: Adjust the ratios of the other four time curves relative to the time curve for the stool sample measured first after stool collection

As shown in Table 2 and FIG. 4A to FIG. 4E, the stool sample that was measured first in the laboratory after defecation, i.e., stool collection in the toilet, corresponds to the data of Subject A's Type 4 stool collected on Day 4 and weighing 20.67 g (purple graph). Using the time curve for that stool sample as a reference, the ratios of the time curves for the other four stool samples were adjusted to determine whether the five Type 4 stool time curves overlap into a single time curve.

The method of adjusting the ratios of the other four curves relative to the time curve for the stool sample measured first after stool collection is as follows.

For example, Subject A's Type 4 stool collected on Day 2 and weighing 27.4 g (red graph) was measured in the laboratory 24 minutes after stool collection. Accordingly, at 24 minutes after stool collection, the rate of gas decrease for Subject A's Type 4 stool weighing 27.4 g (red graph) was 100%, whereas the rate for Subject A's reference Type 4 stool weighing 20.67 g (purple graph) was 45%. Since the time curve for Subject A's Type 4 stool weighing 20.67 g (purple graph), which was measured first after stool collection, served as the reference for ratio adjustment, the time curve for Subject A's Type 4 stool weighing 27.4 g (red graph) was multiplied by 45/100 to determine whether the two curves overlapped into a single time curve. Through the same process, the data of the other stool samples were adjusted relative to the reference stool data (purple graph) to output time curves (see FIG. 4A to FIG. 4E).
Process 5: Confirm whether a single curve is obtained

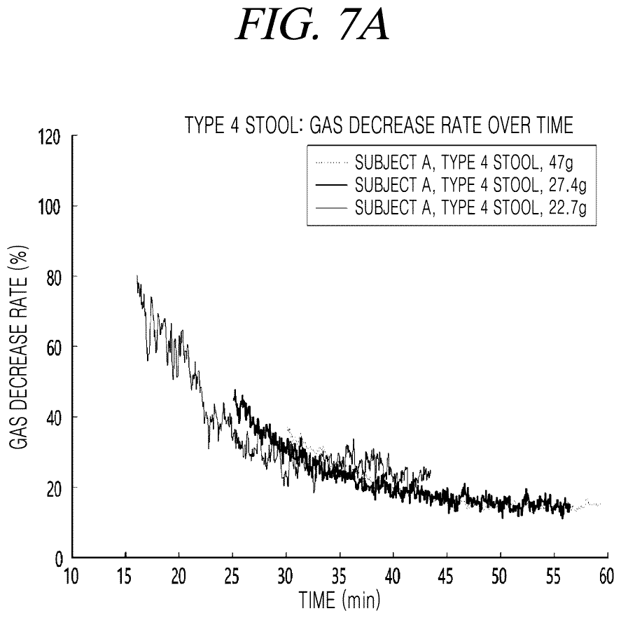

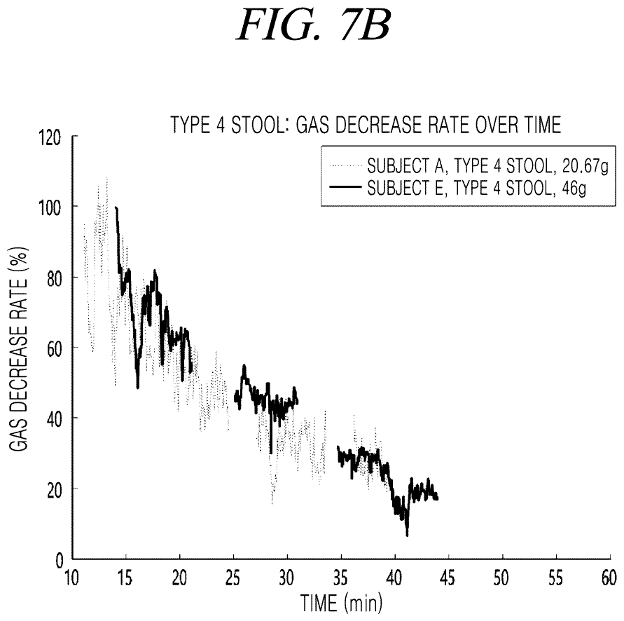

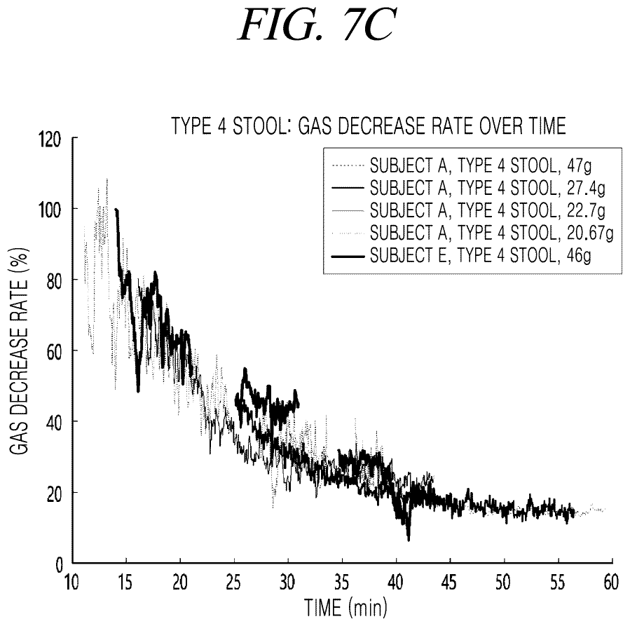

By adjusting the ratios of the respective stool time curves as described above, all the five Type 4 stool time curves overlapped into a single time curve (see FIG. 7A to FIG. 7C).

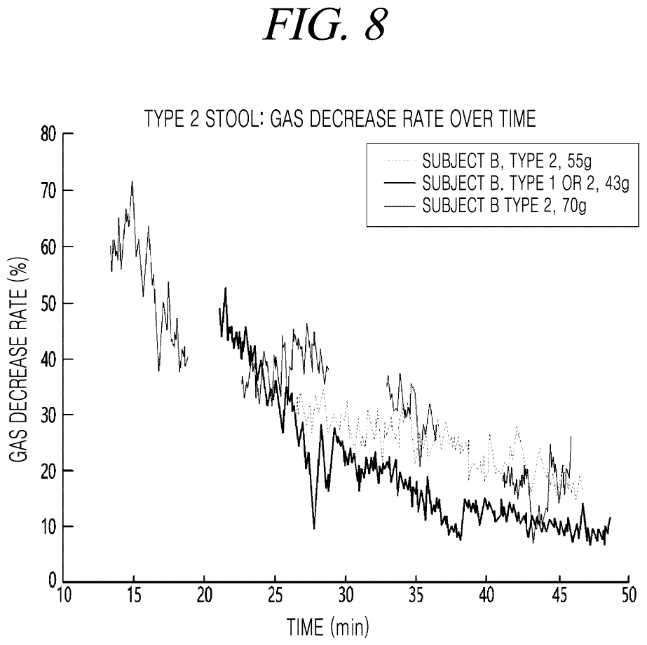

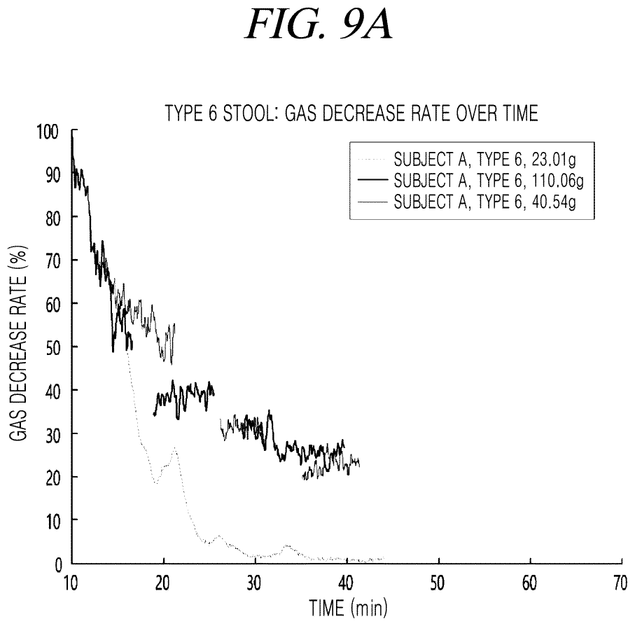

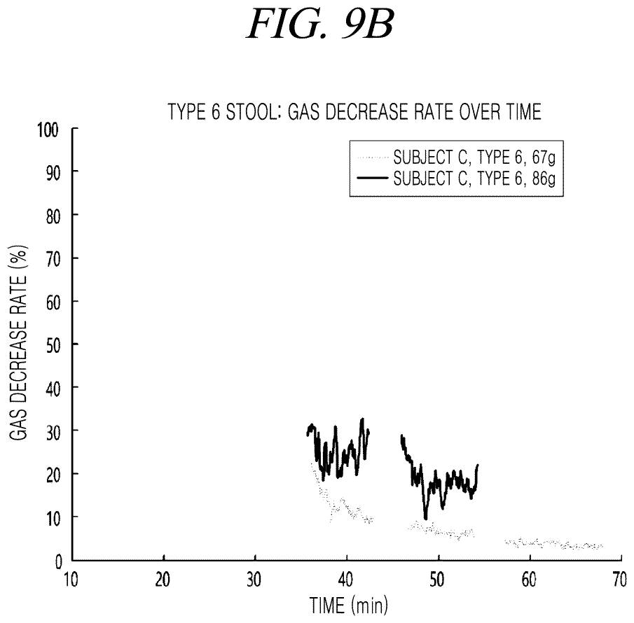

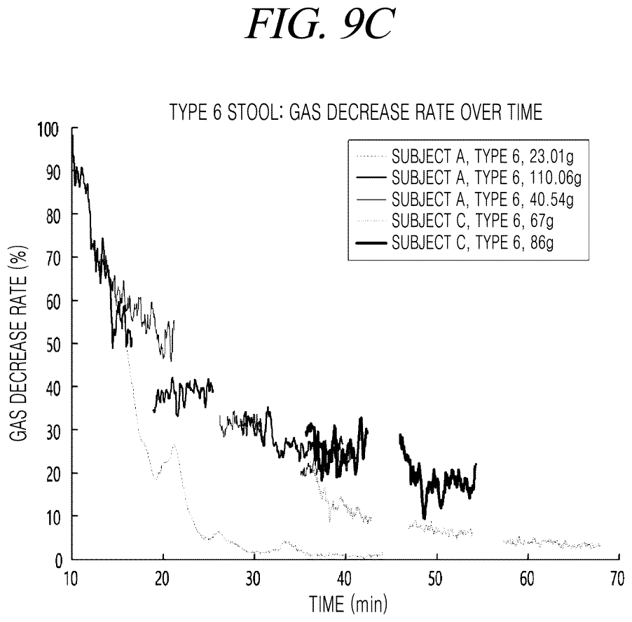

In the same manner, time curve analyses were conducted for Type 2 (FIG. 8) and Type 6 stool samples (FIG. 9A to FIG. 9C). As a result, unique time curves specific to each stool type were confirmed. The time curves for the Type 6 stool samples were found to differ depending on whether spicy food had been consumed.

This confirmed that when a plurality of time curves overlapped into one graph after being arranged based on the stool sample measured first after stool collection, the overlapping stool samples were of the same type.

### (3) Output of Single Type of Time Curve Using Each of Seven Sensors: Analysis of Seven Gas Concentration Graphs Per Stool Sample

In this analysis, time curves were derived from gas concentration graphs for each sensor. When a fecal gas from one stool sample was measured, seven gas concentration graphs were output from the seven sensors and individually converted into time curves. That is, a stool time curve is output for each sensor, and as in the above-described test, the time curves for stool samples of the same type are time-shifted in consideration of the measurement time points after stool collection, and then, the time curves for the other stool samples are arranged and overlapped with the time curve for the stool sample measured first after stool collection for comparative analysis.

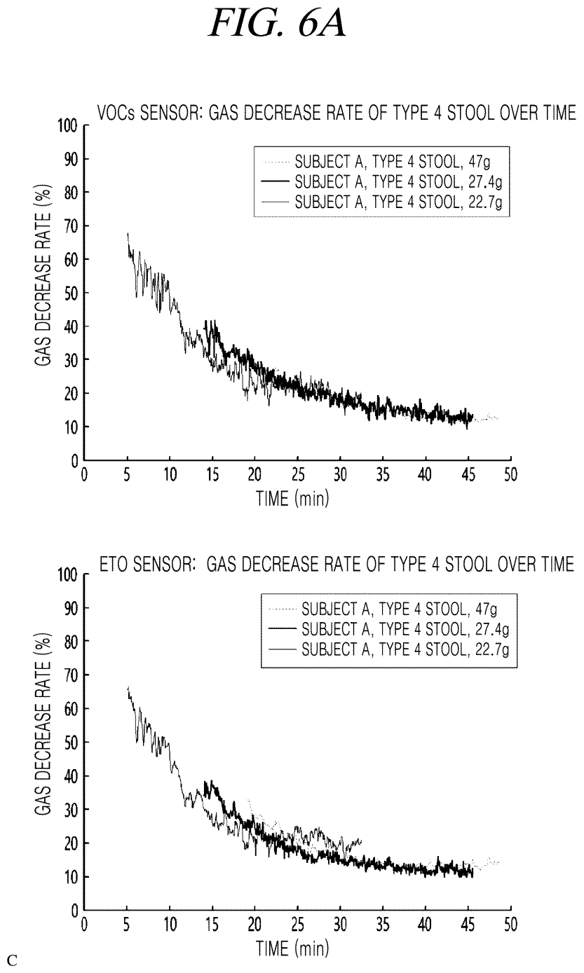

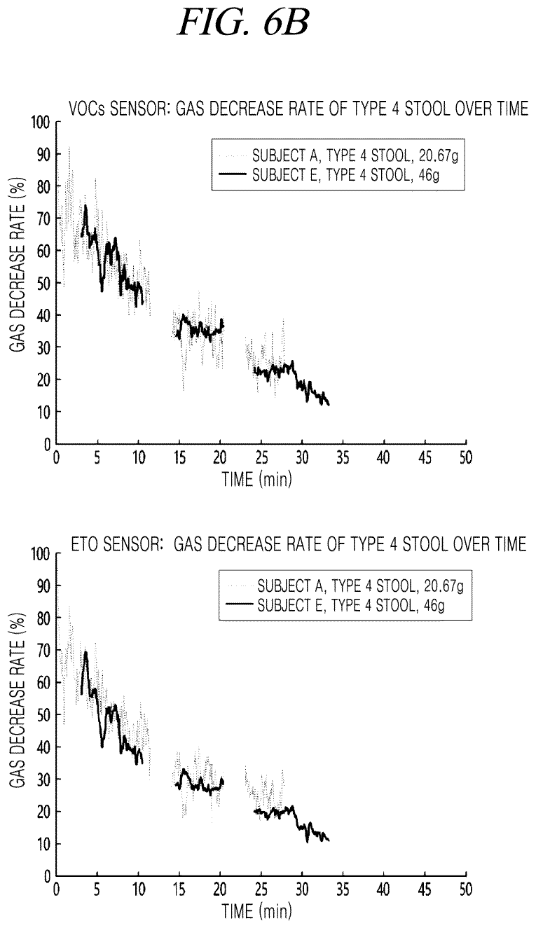

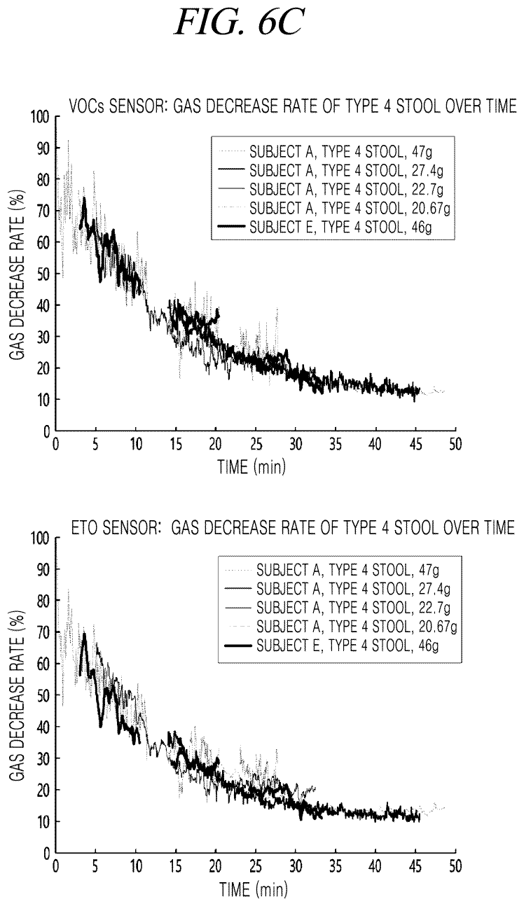

For example, the five Type 4 stool samples were measured by the VOCs sensor, and the five resulting time curves were time-shifted in consideration of the measurement time points after stool collection and then adjusted in ratio based on the time curve for the stool sample measured first. As shown in FIG. 6A to FIG. 6C, the time curves for the five Type 4 stool samples measured with the VOCs sensor overlapped into a single curve.

The analysis confirmed that each of the seven sensors output a single time curve for one stool type (see FIG. 6A to FIG. 6C).

### (4) Output and Comparison of Various Types of Time Curves

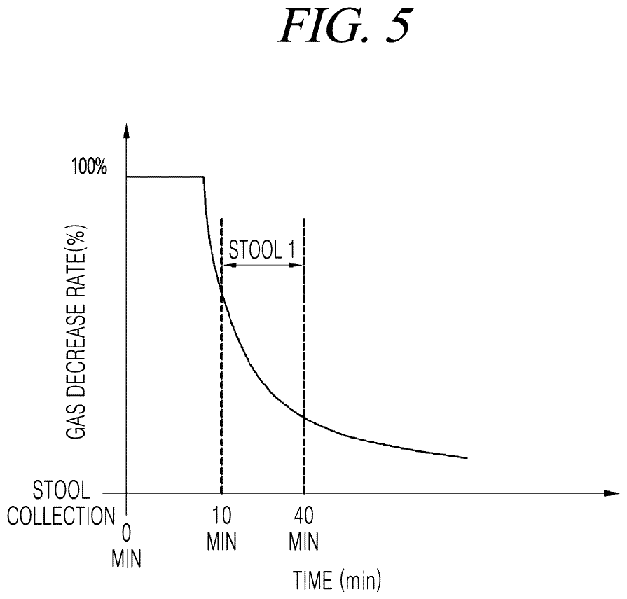

To verify the existence of unique time curves for each stool type, for each stool type, the time curve was derived by setting a point at which the stool sample measured first after stool collection began to decrease to 100%. However, since the gas concentration is highest immediately after stool collection in the toilet, the expected time curve was also derived by setting the gas concentration measured right after stool collection to 100%, and the gas decrease rate graphs for each stool type were compared (see FIG. 5). The total gas concentration graph, obtained by summing the real-time measurement values from all the seven sensors, was used for analysis, rather than the individual sensor graphs. FIG. 5 shows the time curve for one of the five Type 4 stool samples in consideration of the measurement time point after stool collection.

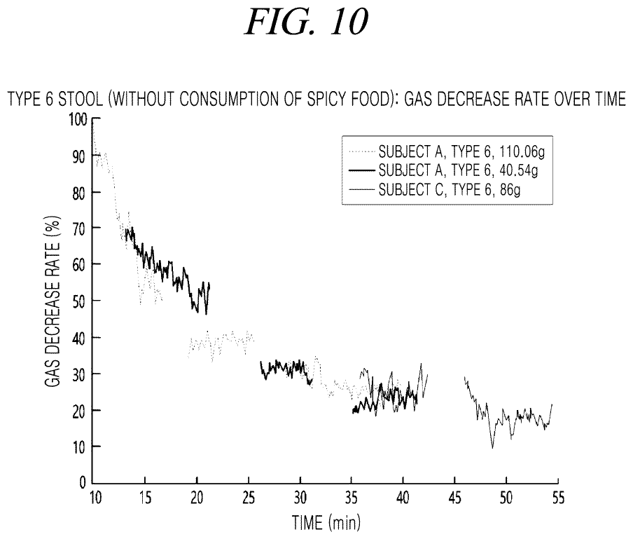

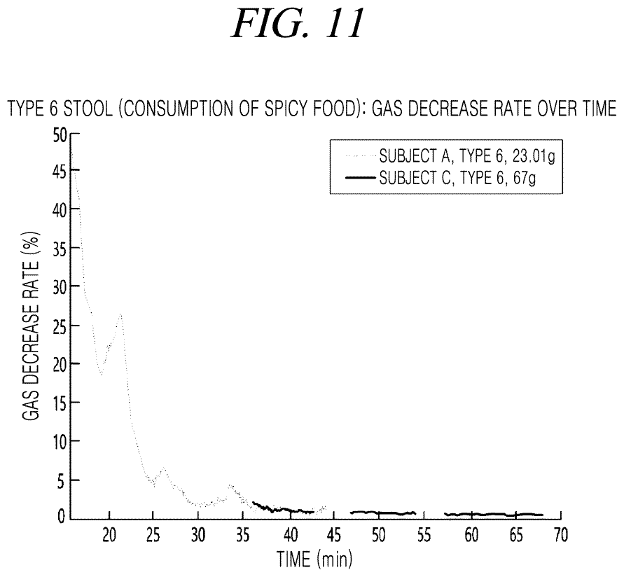

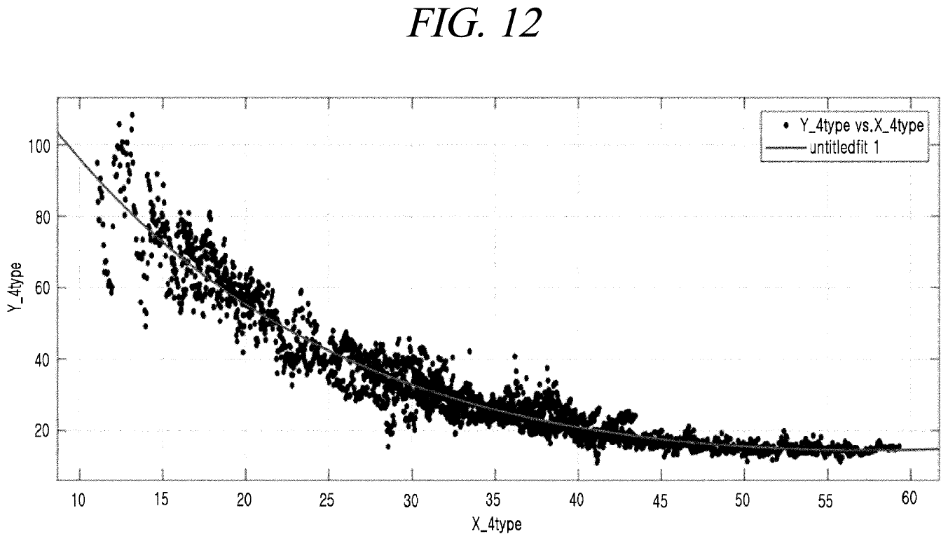

However, the above-described fecal gas measurement method involves transporting stool samples from the toilet to the laboratory, and, thus, it is difficult to perform measurement immediately after stool collection. Therefore, the collected stool data lacked information for a period from stool collection to the start of measurement in the laboratory (stool transport period). To estimate the lacking data, the measured and overlapped time curves for each stool type (FIG. 7A to FIG. 7C, FIG. 8, FIG. 10, and FIG. 11) were curve-fitted to derive the corresponding equations (see FIG. 12).

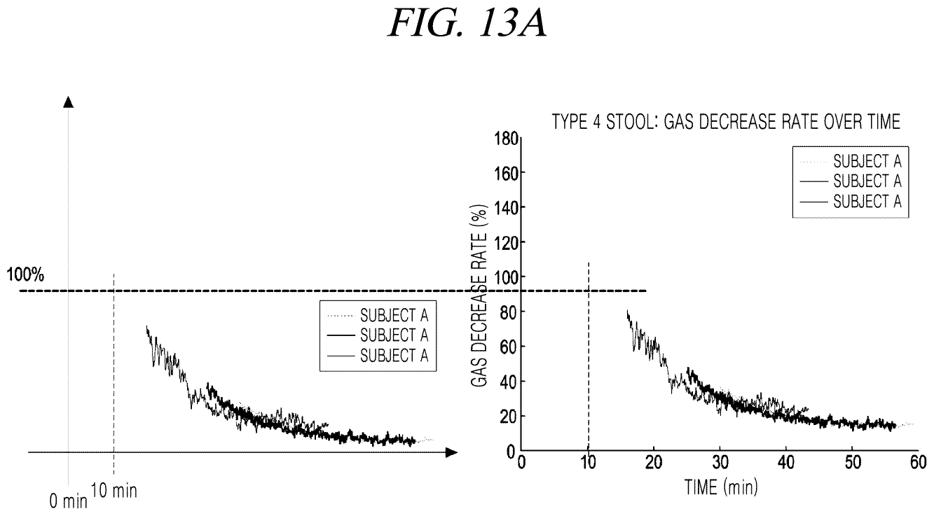

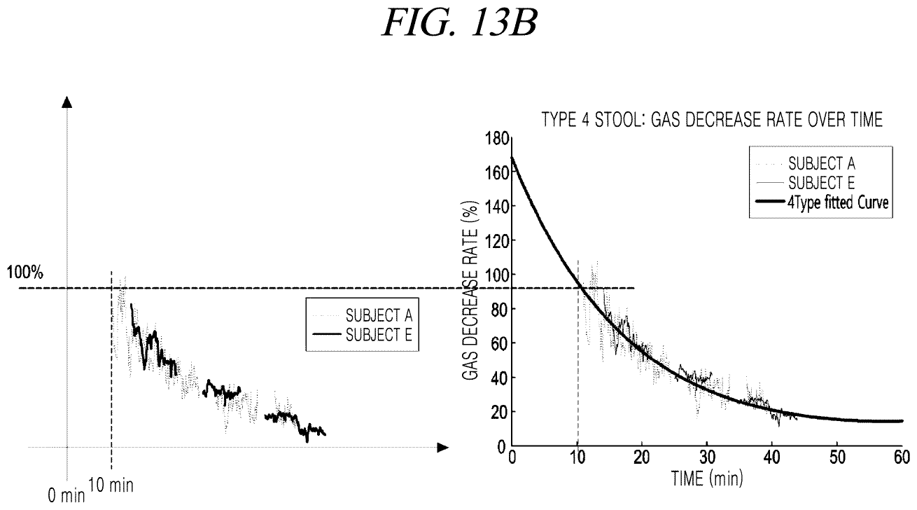

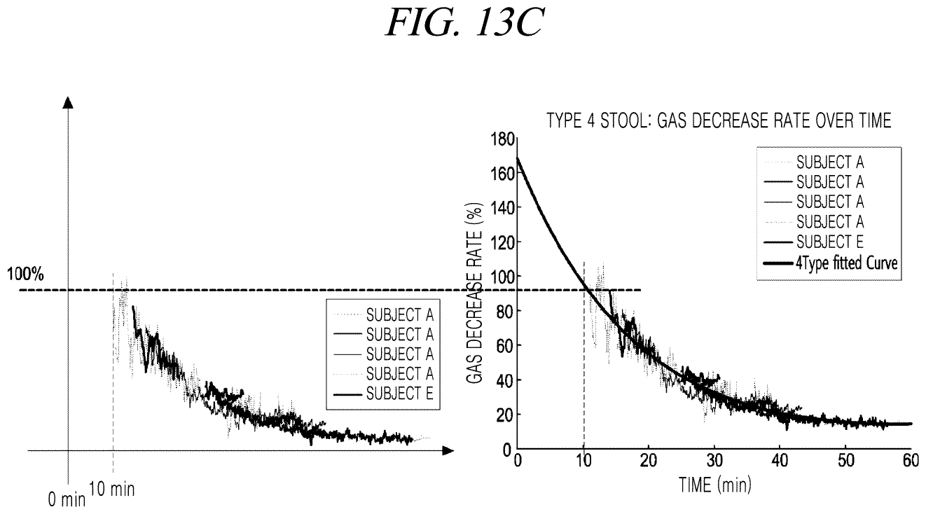

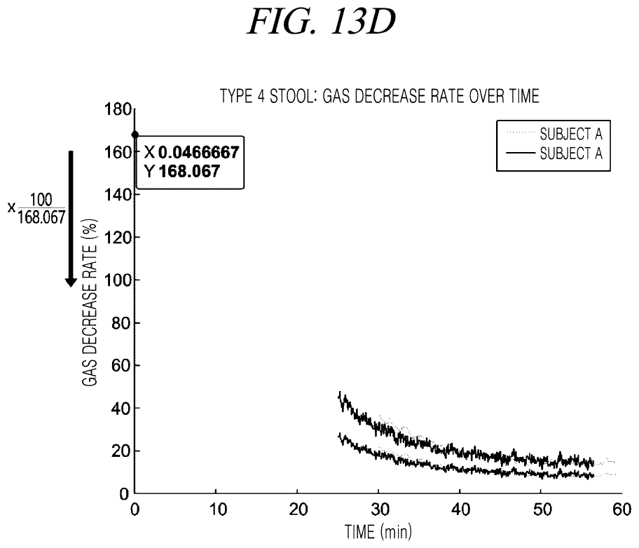

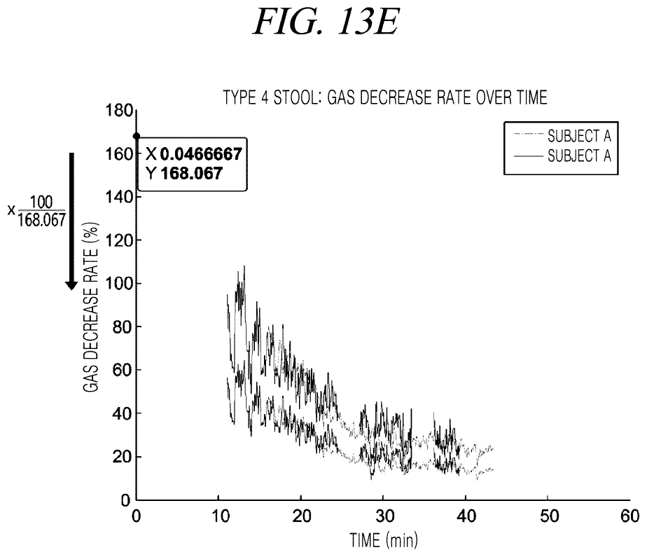

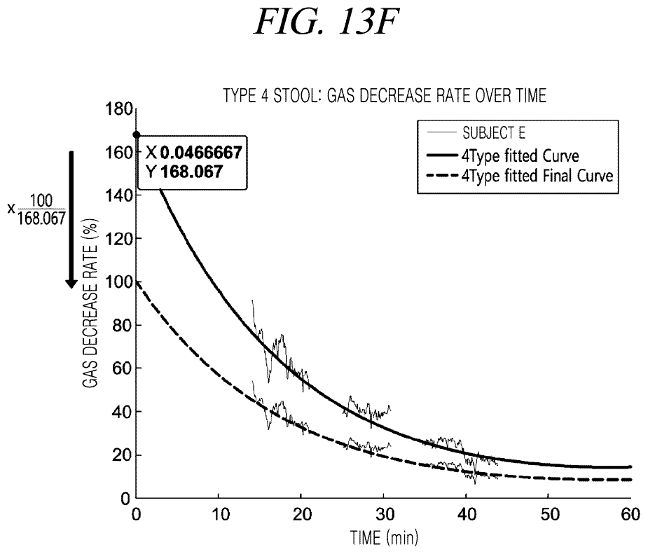

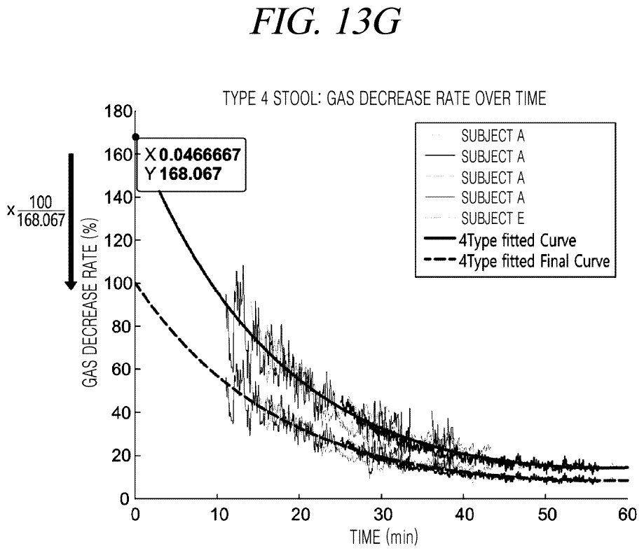

The time curve equations for each stool type were derived by curve-fitting the time curves (FIG. 7A to FIG. 7C, FIG. 8, FIG. 10, and FIG. 11) in which the point at which the stool sample measured first after stool collection began to decrease was set to 100%. Then, to set the rate of gas decrease immediately after stool collection (0 min) to 100% in the derived time curve equations, the time curve equations were adjusted as described below to derive inherent and complete time curves for each stool type. The corresponding analysis processes are shown in FIG. 13A to FIG. 13G.

$$\begin{matrix}INHERENT AND COMPLETE TIME \\ CURVE EQUATION FOR TYPE 4 STOOL\end{matrix} =$$

$$TYPE 4 STOOL TIME CURVE EQUATION ( 1 ) X \frac{100}{GAS AMOUNT ( \% ) AT 0 MINUTES IN EQUATION ( 1 )}$$

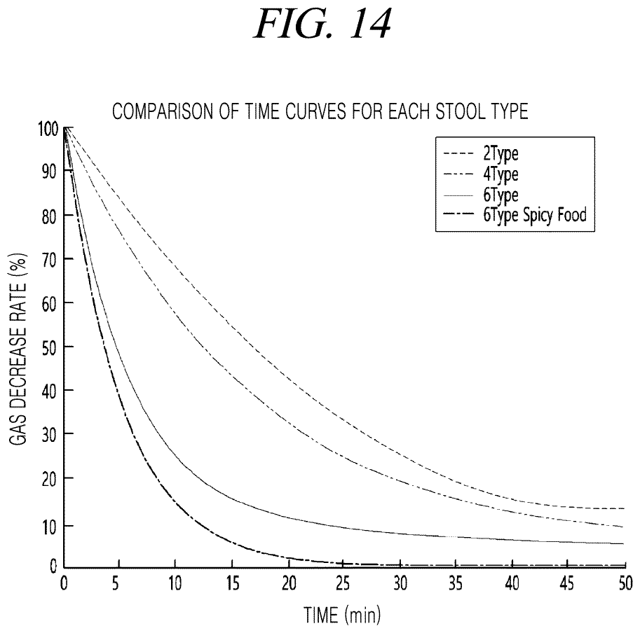

Through this process, gas decrease rate graphs were derived for each stool type (see FIG. 10). The analysis result confirmed that each stool type possesses an inherent and complete time curve, exhibiting distinct rates and trends of gas decrease over time (see FIG. 14).

The analysis result confirmed unique gas decrease rate graphs (time curves) exist for each stool type and that the rates and trends of gas decrease over time can be identified for each stool type.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description. It shall be understood that all modifications and embodiments conceived from the meaning and scope of the claims and their equivalents are included in the scope of the present disclosure.

We claim:

1. A method for analyzing fecal gas time curves of the same type for Bristol stool type classification and body condition analysis, comprising:
a process (a) of measuring concentration values (ppm) of gases emitted from each stool sample using gas sensors;
a process (b) of summing the measured gas concentration values in real time to derive a total gas concentration graph for each stool sample;
a process (c) of setting a point at which the gas begins to decrease in the derived total gas concentration graph to 100% and deriving a time curve for each stool sample;
a process (d) of arranging the derived time curves for the respective stool samples according to a sequence of measurement time points after stool collection;
a process (e) of overlapping the time curves for the other stool samples based on a time curve for the stool sample measured first after stool collection to output a single time curve; and
a process (f) of confirming that data of the stool samples of the same type overlap into a single time curve.

2. The fecal gas time curve analysis method of claim 1,
wherein the process (a) includes:
a process of placing a prepared stool sample;
a process of turning on a gas sensor switch to start data acquisition and turning on a fan within two minutes to circulate air inside the gas sensor;
a process of placing the prepared stool sample on a stool collection paper within two minutes, closing a toilet lid to achieve maximum sealing from the outside, and attaching a connection tube to connect the toilet to the gas sensor;
a process of measuring a fecal gas for 20 minutes to 40 minutes and then disconnecting the tube; and
a process of turning off the gas sensor switch within two minutes to stop data transmission.

3. The fecal gas time curve analysis method of claim 1,
wherein the gas sensor used in the process (a) includes one or more selected from the group consisting of SO₂, VOCs, CH₂O, ETO, C₂H₄, H₂S and NH₃ gas sensors.

4. A fecal gas time curve analysis method for Bristol stool type classification and body condition analysis, comprising:
a process (a) of measuring concentration values (ppm) of gases emitted from each stool sample using gas sensors;
a process (b) of summing the measured gas concentration values in real time to derive a total gas concentration graph for each stool sample;
a process (c) of setting a point at which the gas begins to decrease in the derived total gas concentration graph to 100% and deriving a time curve for each stool sample;
a process (d) of arranging the derived time curves for the respective stool samples according to a sequence of measurement time points after stool collection;
a process (e) of overlapping the time curves for the other stool samples based on a time curve for the stool sample measured first after stool collection to output a single time curve;
a process (f) of confirming that data of the stool samples of the same type overlap into a single time curve;
a process (g) of performing curve fitting on the overlapped time curve for the stool samples of the same type, as confirmed above, to derive a time curve equation;
a process (h) of deriving a single type of time curve, in which the amount of gas measured immediately after stool collection is set to 100%, through a calculation of
$$TIME CURVE EQUATION AS DERIVED BY ABOVE X \frac{100}{GAS AMOUNT ( \% ) AT 0 MINUTES IN THE TIME CURVE EQUATION} ;$$
and
a process (i) of comparing the derived time curve with another type of time curve.

5. The fecal gas time curve analysis method of claim 4,
wherein the process (a) includes:
a process of placing a prepared stool sample;
a process of turning on a gas sensor switch to start data acquisition and turning on a fan within two minutes to circulate air inside the gas sensor;
a process of placing the prepared stool sample on a stool collection paper within two minutes, closing a toilet lid to achieve maximum sealing from the outside, and attaching a connection tube to connect the toilet to the gas sensor;
a process of measuring a fecal gas for 20 minutes to 40 minutes and then disconnecting the tube; and
a process of turning off the gas sensor switch within two minutes to stop data transmission.

6. The fecal gas time curve analysis method of claim 4,
wherein the gas sensor used in the process (a) includes one or more selected from the group consisting of SO₂, VOCs, CH₂O, ETO, C₂H₄, H₂S and NH₃ gas sensors.

7. A fecal gas time curve output method for Bristol stool type classification and body condition analysis, comprising:
a process (a) of measuring concentration values (ppm) of respective gases emitted from a single stool sample using gas sensors;
a process (b) of deriving time curves for the respective gases by setting a point at which the measured gas concentration values begin to decrease to 100%; and
a process (c) of arranging the derived time curves for the respective gases according to a sequence of measurement time points after stool collection.

8. The fecal gas time curve output method of claim 7,
wherein the process (a) includes:
a process of placing a prepared stool sample;
a process of turning on a gas sensor switch to start data acquisition and turning on a fan within two minutes to circulate air inside the gas sensor;
a process of placing the prepared stool sample on a stool collection paper within two minutes, closing a toilet lid to achieve maximum sealing from the outside, and attaching a connection tube to connect the toilet to the gas sensor;
a process of measuring a fecal gas for 20 minutes to 40 minutes and then disconnecting the tube; and
a process of turning off the gas sensor switch within two minutes to stop data transmission.

9. The fecal gas time curve output method of claim 7,
wherein the gas sensor used in the process (a) includes one or more selected from the group consisting of SO₂, VOCs, CH₂O, ETO, C₂H₄, H₂S and NH₃ gas sensors.
